(19) United States
(12) Patent Application Publication
(10) Pub. No.: US 2019/0201096 A1
(43) Pub. Date: Jul. 4, 2019

(54) Controlled photomechanical and photothermal tissue treatment in the picosecond regime

(71) Applicant: Cynosure, LLC, Westford, MA (US)

(72) Inventors: Richard Shaun Welches, Manchester, NH (US); Mirko Georgiev Mirkov, Chelmsford, MA (US)

(21) Appl. No.: 15/932,451

(22) Filed: Aug. 20, 2018

Related U.S. Application Data
(63) Continuation of application No. 14/678,210, filed on Apr. 3, 2015, now abandoned.
Application No. 14/678,210 is a continuation-in-part of application No. 14/209,270, filed on Mar. 13, 2014.
(60) Provisional application No. 61/974,784, filed on Apr. 3, 2014.
(60) Provisional application No. 61/779,411, filed on Mar. 13, 2013.
(60) Provisional application No. 61/909,563, filed on Nov. 27, 2013.

(51) Int. Cl. A61B 18/20; B23K 26/0622; A61B 18/26

(52) U.S. Cl. CPC A61B 18/20; B23K 26/0624; A61B 2018/00702; A61B 2018/263; A61B 18/26

(57) ABSTRACT

Systems and methods for treating tissue by directing light pulses using bubbles generating in tissue using previously transmitted light pulses are disclosed. Systems and methods for treating tissue using a lens array comprising a pitch or separation distance sized to overlap sonoporation induced shockwaves are also disclosed. In one embodiment, the shockwaves are generated in response to incident light pulses directed through adjacent lenses in the array. Systems and methods can improve porosity of the cellular membrane. Systems and methods for creating channels in tissue by using stacked pulses are also disclosed.

### CROSS-REFERENCE TO RELATED APPLICATIONS

This application is a continuation-in-part of and incorporates by reference the entire contents of U.S. Provisional Application No. 61/779,411 filed on Mar. 13, 2013 entitled “Picosecond Laser Induced Optical Breakdown Therapy and Method for the In Vivo Rejuvenation of Tissues,” U.S. Provisional Application No. 61/909,563 filed on Nov. 27, 2013 entitled “Controlled Photomechanical and Photothermal Tissue Treatment in the Picosecond Regime,” and U.S. application Ser. No. 14/209,270 filed on Mar. 13, 2014 entitled “Controlled Photomechanical and Photothermal Tissue Treatment in the Picosecond Regime” Published on Mar. 19, 2015 as U.S. Publication No. US-2015-0080863-A1, and claims priority to U.S. Provisional Application No. 61/974,784 filed on Apr. 3, 2014 entitled “Controlled Photomechanical and Photothermal Tissue Treatment in the Picosecond Regime”, the entirety of which is herein incorporated by reference.

### FIELD OF THE INVENTION

The present disclosure relates to an apparatus and methods for delivering laser energy having a short pulse duration (e.g., less than about 1 nanosecond) and high energy output per pulse into tissues, resulting in tissue damage and tissue remodeling and regeneration.

### BACKGROUND

Photothermal mechanisms for tissue treatment have been widely exploited for medical and cosmetic tissue treatments including dermatology treatments. Currently available light based (including laser) treatments for conditions such as scar modification rely on relatively aggressive thermal treatment. In order to achieve certain treatments at desired depths the level of photothermal temperature rise necessary as part of a desired treatment can result in unwanted/undesirable additional thermal damage to adjacent regions. In treating such medical and cosmetic conditions it is desirable to limit thermal damage to the target treatment area and avoid unnecessary thermal damage to areas outside the target treatment area.

### SUMMARY OF THE INVENTION

The present disclosure generally relates to a system for tissue treatment. The system includes an optical system having at least one foci for concentrating a laser emission to at least one target at a depth in the tissue at a fluence ranging from about 0.8 J/cm2 to about 50 J/cm2 at a pulse width. The fluence and the pulse width are selected to exceed an electron ionization threshold of the target to result in an ablation volume of at least a portion of the target. The pulse width is selected to control a pressure wave emission from the ablation volume to tissue adjacent the target. The system controls a firing time between a first pulse and a second pulse. The pulse width can be within the range of from about 260 picoseconds to about 900 picoseconds or from about 260 picoseconds to about 500 picoseconds.

The system can further include a controller for tuning the pulse width, whereby tuning the controller to a different pulse width changes the ratio of the pressure wave to a thermal effect on the tissue adjacent the target. Alternatively or in addition, the system can include a controller for tuning the firing time between the first pulse and the second pulse. Alternatively or in addition, the system can include controller for tuning the pulse width whereby tuning the controller changes the firing time between the first pulse and the second pulse.

In one embodiment, the controller triggers firing of the first pulse of the laser and triggers the firing of the second pulse of the laser through one or more bubbles generated in a target material in response to the first pulse. Optionally, the second pulse is fired through a bubble in a post-ionized state. In some embodiments, the firing time is selected to correspond to a bubble existence time.

In another aspect, the disclosure relates generally to, a method for tissue treatment, that includes, providing a laser having a pulse width ranging and a fluence ranging from about 0.8 J/cm2 to about 50 J/cm2, concentrating a first laser emission to target at least a first depth in the tissue such that a first sonoporation induced shockwave results, concentrating a second laser emission to target at least a second depth in the tissue such that a second sonoporation induced shockwave results, and overlapping the first sonoporation induced shockwave and the second sonoporation induced shockwave. In one embodiment, the second depth achieved by the treatment method is deeper than the first depth. In some embodiments, overlapping the first laser emission and the second laser emission creates a channel in the tissue. The pulse width may be controlled to provide a pressure wave emission from the ablation volume to tissue adjacent the target. In some embodiments, the method includes controlling the firing time between the first laser emission and the second laser emission. The pulse width can range from about 260 picoseconds to about 900 picoseconds, or from about 260 picoseconds to about 500 picoseconds.

In still another aspect, the disclosure relates to a method for tissue treatment including transmitting a first light pulse to a first treatment region, transmitting a second light pulse to a second treatment region, generating a first shockwave at the first treatment region and generating a second shockwave at the second treatment region, the second treatment region a distance p from the first treatment region and overlapping the first shock wave and the second shockwave. The distance p may be less than about 400 microns. In one embodiment, the pressure of the first shockwave and the second shockwave is less than about 5 psi. In another embodiment, the pressure of the first shockwave and the second shockwave ranges from about 1.5 psi to about 3 psi. The method can include changing a porosity of a membrane disposed in proximity to the first and the second shockwaves. The method can include controlling the firing time between transmitting the first light pulse and the second light pulse.

In accordance with the embodiments of this disclosure, the optical emission or light pulse is a laser pulse that targets one or more of a blood cell, hemoglobin, or melanin. For example, in one embodiment, the laser pulse has a wavelength of about 755 nm and the target is a blood cell.

### BRIEF DESCRIPTION OF THE FIGURES

FIG. 1, in a schematic diagram, illustrates an exemplary system having a wavelength-shifting resonator for generating picosecond pulses in accordance with various aspects of the applicants' teachings.

FIG. 2 illustrates a tissue injury caused by a picosecond laser including an ablation volume of a cavitation bubble with a layer of tissue adjacent the cavitation bubble being subjected to relatively intense pressure and the next progressively outer layer(s) of tissue being subjected to relatively less intense pressure.

FIG. 3, in a schematic diagram, illustrates two exemplary lens arrays suitable for directing light pulses in accordance with various aspects of the applicants' teachings.

FIGS. 4A-4B are images illustrating bubbles generated in response to a light pulse suitable for focusing one or more subsequent light pulses in accordance with various aspects of the applicants' teachings.

FIGS. 5(a)-5(b) illustrate a sequential one two pulse arrangement for a picosecond drive pulse that is split by an adjustable beam splitter such that one split part can be delayed by an adjustable amount of time such that the delayed part arrives at the target while the target area is still ionized by the first non-delayed part and this can act to enable the second part to be immediately and fully absorbed by the target area and acts to drive a second pulse of expansion.

FIGS. 5(c)-5(d) illustrate that the point where the peak pressure provided by the first pulse is just beginning to wane (but is still in a plasma/ionized state) then consequently the initial shockwaves generated by the first pulse will detach (such that it is no longer driven by the ablation bubble expansion) and will begin to propagate into the tissue just as the second pulse arrives this can provide an enhanced lesion.

### DETAILED DESCRIPTION

The present disclosure relates to laser systems having sub-nanosecond pulsing (e.g., picosecond pulsing). Exemplary systems are described in our U.S. Pat. Nos. 7,929,579 and 7,586,957, both incorporated herein by reference. These patents disclose picosecond laser apparatuses and methods for their operation and use. Herein we describe certain improvements to such systems.

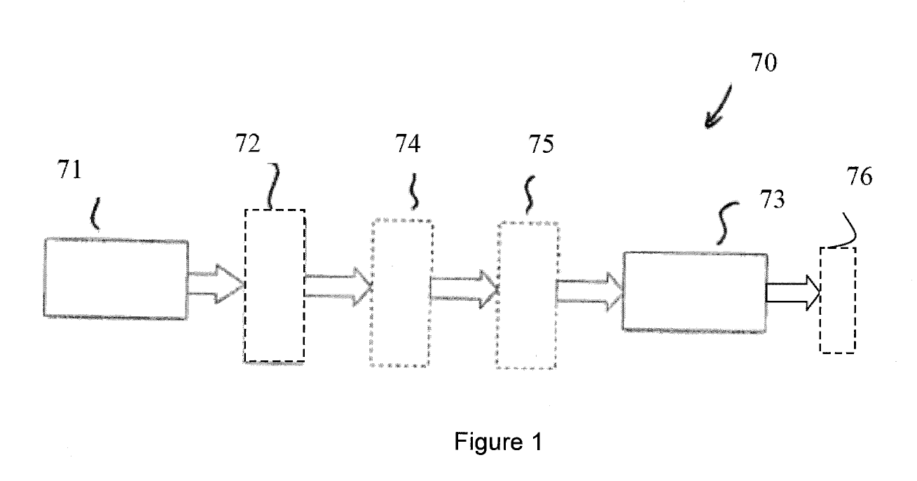

With reference now to FIG. 1, an exemplary system 70 for the generation and delivery of picosecond-pulsed treatment radiation is schematically depicted. As shown in FIG. 1, the system generally includes a pump radiation source 71 for generating picosecond pulses at a first wavelength and a treatment beam delivery system 73 for delivering a pulsed treatment beam to the patient's skin.

The system optionally includes a wavelength-shifting resonator 72 for receiving the picosecond pulses generated by the pump radiation source 71 and emitting radiation at a second wavelength in response thereto to the treatment beam delivery system 73.

The pump radiation source 71 generally generates one or more pulses at a first wavelength to be transmitted to the wavelength-shifting resonator 72, and can have a variety of configurations. For example, the pulses generated by the pump radiation source 71 can have a variety of wavelengths, pulse durations, and energies. In some aspects, as will be discussed in detail below, the pump radiation source 71 can be selected to emit substantially monochromatic optical radiation having a wavelength that can be efficiently absorbed by the wavelength-shifting resonator 72 in a minimum number of passes through the gain medium. Additionally, it will be appreciated by a person skilled in the art in light of the present teachings that the pump radiation source 71 can be operated so as to generate pulses at various energies, depending for example, on the amount of energy required to stimulate emission by the wavelength-shifting resonator 72 and the amount of energy required to perform a particular treatment in light of the efficiency of the system 70 as a whole.

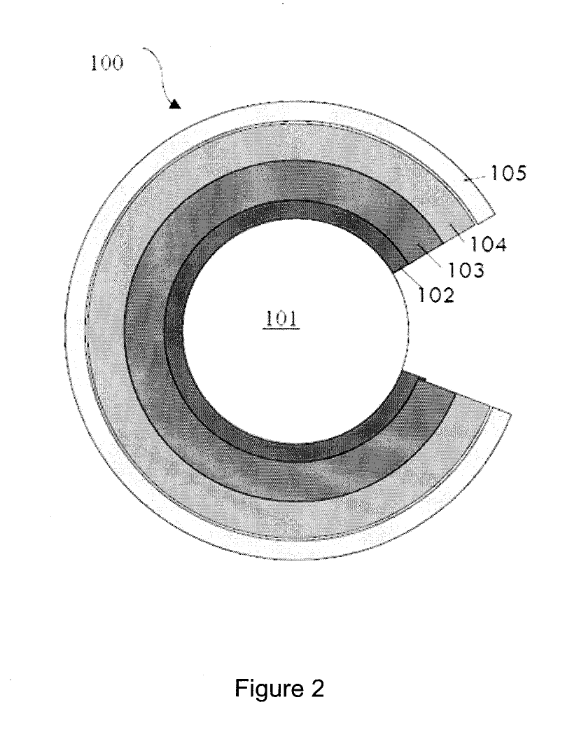

In various aspects, the pump radiation source 71 can be configured to generate picosecond pulses of optical radiation. That is, the pump radiation source can generate pulsed radiation exhibiting a pulse duration less than about 1000 picoseconds (e.g., within a range of about 500 picoseconds to about 800 picoseconds). In an exemplary embodiment, the pump radiation source 71 for generating the pump pulse at a first wavelength can include a resonator (or laser cavity containing a lasing medium), an electro-optical device (e.g., a Pockels cell), and a polarizer (e.g., a thin-film polarizer), as described for example with reference to FIG. 2 of U.S. Pat. No. 7,586,957, issued on Sep. 8, 2009 and entitled “Picosecond Laser Apparatus and Methods for Its Operation and Use,” the contents of which are hereby incorporated by reference in its entirety.

In an exemplary embodiment, the lasing or gain medium of the pump radiation source 71 can be pumped by any conventional pumping device such as an optical pumping device (e.g., a flash lamp) or an electrical or injection pumping device. In an exemplary embodiment, the pump radiation source 71 comprises a solid state lasing medium and an optical pumping device. Exemplary solid state lasers include an alexandrite or a titanium doped sapphire (TIS) crystal, Nd:YAG lasers, Nd:YAP, Nd:YAlO3 lasers, Nd:YAF lasers, and other rare earth and transition metal ion dopants (e.g., erbium, chromium, and titanium) and other crystal and glass media hosts (e.g., vanadate crystals such as YVO4, fluoride glasses such as ZBLN, silica glasses, and other minerals such as ruby).

At opposite ends of the optical axis of the resonator can be first and second mirrors having substantially complete reflectivity such that a laser pulse traveling from the lasing medium towards second mirror will first pass through the polarizer, then the Pockels cell, reflect at second mirror, traverse Pockels cell a second time, and finally pass through polarizer a second time before returning to the gain medium. Depending upon the bias voltage applied to the Pockels cell, some portion (or rejected fraction) of the energy in the pulse will be rejected at the polarizer and exit the resonator along an output path to be transmitted to the wavelength-shifting resonator 72. Once the laser energy, oscillating in the resonator of the pump radiation source 71 under amplification conditions, has reached a desired or maximum amplitude, it can thereafter be extracted for transmission to the wavelength-shifting resonator 72 by changing the bias voltage to the Pockels cell such that the effective reflectivity of the second mirror is selected to output laser radiation having the desired pulse duration and energy output.

The wavelength-shifting resonator 72 can also have a variety of configurations in accordance with the applicant's present teachings, but is generally configured to receive the pulses generated by the pump radiation source 71 and emit radiation at a second wavelength in response thereto. In an exemplary embodiment, the wavelength-shifting resonator 72 comprises a lasing medium and a resonant cavity extending between an input end and an output end, wherein the lasing medium absorbs the pulses of optical energy received from the pump radiation source 71 and, through a process of stimulated emission, emits one or more pulses of optical laser radiation exhibiting a second wavelength.

As will be appreciated by a person skilled in the art in light of the present teachings, the lasing medium of the wavelength-shifting resonator can comprise a neodymium-doped crystal, including by way of non-limiting example solid state crystals of neodymium-doped yttrium-aluminum garnet (Nd:YAG), neodymium-doped pervoskite (Nd:YAP or Nd:YAlO3), neodymium-doped yttrium-lithium-fluoride (Nd:YAF), and neodymium-doped vanadate (Nd:YVO4) crystals. It will also be appreciated that other rare earth transition metal dopants (and in combination with other crystals and glass media hosts) can be used as the lasing medium in the wavelength-shifting resonator. Moreover, it will be appreciated that the solid state laser medium can be doped with various concentrations of the dopant so as to increase the absorption of the pump pulse within the lasing medium. By way of example, in some aspects the lasing medium can comprise between about 1 and about 3 percent neodymium.

The lasing medium of the wavelength-shifting resonator 72 can also have a variety of shapes (e.g., rods, slabs, cubes) but is generally long enough along the optical axis such that the lasing medium absorbs a substantial portion (e.g., most, greater than 80%, greater than 90%) of the pump pulse in two passes through the crystal. As such, it will be appreciated by a person skilled in the art that the wavelength of the pump pulse generated by the pump radiation source 71 and the absorption spectrum of the lasing medium of the resonator 72 can be matched to improve absorption. However, whereas prior art techniques tend to focus on maximizing absorption of the pump pulse by increasing crystal length, the resonator cavities disclosed herein instead utilize a short crystal length such that the roundtrip time of optical radiation in the resonant cavity (i.e.,

$${t}_{roundtrip} = 2 \frac{n \cdot {l}_{resonator}}{c} ,$$

where n is the index of refraction of the lasing medium and c is the speed of light) is substantially less than the pulse duration of the input pulse (i.e., less than the pulse duration of the pulses generated by the pump radiation source 71). For example, in some aspects, the roundtrip time can be less than 5 times shorter than the duration of the picosecond pump pulses input into the resonant cavity (e.g., less than 10 times shorter). Without being bound by any particular theory, it is believed that by shortening the resonant cavity, the output pulse extracted from the resonant cavity can have an ultra-short duration without the need for additional pulse-shaping (e.g., without use of a modelocker, Q-switch, pulse picker or any similar device of active or passive type). For example, the pulses generated by the wavelength-shifting resonator can have a pulse duration less than 1000 picoseconds (e.g., about 500 picoseconds, about 750 picoseconds).

After the picosecond laser pulses are extracted from the wavelength-shifting resonator 72, they can be transmitted directly to the treatment beam delivery system 73 for application to the patient's skin, for example, or they can be further processed through one or more optional optical elements shown in phantom, such as an amplifier 74, frequency doubling waveguide 75, and/or filter (not shown) prior to being transmitted to the treatment beam delivery system. As will be appreciated by a person skilled in the art, any number of known downstream optical (e.g., lenses) electro-optical and/or acousto-optic elements modified in accordance with the present teachings can be used to focus, shape, and/or alter (e.g., amplify) the pulsed beam for ultimate delivery to the patient's skin to ensure a sufficient laser output, while nonetheless maintaining the ultrashort pulse duration generated in the wavelength-shifting resonator 72. For example an optical element 76 (in phantom) can include one or more foci in, for example, the form of a lens array such as a diffractive lens array.

Lasers are recognized as controllable sources of radiation that are relatively monochromatic and coherent (i.e., have little divergence). Laser energy is applied in an ever-increasing number of areas in diverse fields such as telecommunications, data storage and retrieval, entertainment, research, and many others. In the area of medicine, lasers have proven useful in surgical and cosmetic procedures where a precise beam of high energy radiation causes localized heating and ultimately the destruction of unwanted tissues. Such tissues include, for example, subretinal scar tissue that forms in age-related macular degeneration (AMD) or the constituents of ectatic blood vessels that constitute vascular lesions.

Most of today's aesthetic lasers rely on heat to target tissue and desired results must be balanced against the effects of sustained, elevated temperatures. The principle of selective photothermolysis underlies many conventional medical laser therapies to treat diverse dermatological problems such as unwanted hair, leg veins, port wine stain birthmarks, and other ectatic vascular and pigmented lesions. The tissue layers including the dermal and epidermal layers containing the targeted structures are exposed to laser energy having a wavelength that is preferentially or selectively absorbed in these structures. This leads to localized heating to a temperature that denatures constituent proteins and/or disperses pigment particles (e.g., to about 70 degrees C.). The fluence, or energy per unit area, used to accomplish this denaturation or dispersion is generally based on the amount required to achieve the desired targeted tissue temperature, before a significant portion of the absorbed laser energy is lost to diffusion. The fluence must, however, be limited to avoid denaturing tissues surrounding the targeted area.

Fluence is not the only consideration governing the suitability of laser energy for particular applications. The pulse duration (also referred to as the pulse width) and pulse intensity, for example, can impact the degree to which laser energy diffuses into surrounding tissues during the pulse and/or causes undesired, localized vaporization. In terms of the pulse duration of the laser energy used, conventional approaches have focused on maintaining this value below the thermal relaxation time of the targeted structures, in order to achieve optimum heating. For the small vessels contained in portwine stain birthmarks, for example, thermal relaxation times and hence the corresponding pulse durations of the treating radiation are often on the order of hundreds of microseconds to several milliseconds.

Cynosure's PicoSure™ brand laser system, which entered the commercial market in late March 2013 is the first aesthetic laser system to utilize picosecond technology that delivers laser energy at speeds measured in trillionth of seconds (10-12). An exemplary PicoSure™ brand picosecond laser apparatus is detailed in our U.S. Pat. Nos. 7,586,957 and 7,929,579, the contents of which are incorporated herein by reference. A picosecond laser apparatus provides for extremely short pulse durations, resulting in a different approach to treating various conditions than traditional photothermal-based treatments. Picosecond laser pulses have durations below the acoustic transit time of a sound wave through targeted tissues and are capable of generating both photothermal and photomechanical (e.g., shock wave and/or pressure wave) effects through pressures built up in the target.

Clinical results on tattoo removal with these systems show a higher percentage of ink particle clearance, which is achieved in fewer treatments. PicoSure™ picosecond laser systems can deliver both heat and mechanical stress (e.g., shock waves and/or pressure waves) to shatter the target ink particles from within before any substantial thermal energy can disperse to surrounding tissue. PicoSure picosecond laser systems, employing Pressure Wave™ technology, are useful for other applications including other aesthetic indications such as dermal rejuvenation, as well as other therapeutic applications where an increase in vascularization is desirable.

Blast injuries caused by detonation of explosives are known to cause shock waves and/or pressure waves that cause primary injuries that can damage a person's body including the lung, brain, and/or gut. Primary blast injuries are caused by blast shock waves and/or pressure waves. These are especially likely when a person is close to an exploding munition, such as a land mine. The ears are most often affected by the overpressure, followed by the lungs and the hollow organs of the gastrointestinal tract. Gastrointestinal injuries may present after a delay of hours or even days. Injury from blast overpressure is a pressure and time dependent function. By increasing the pressure or its duration, the severity of injury will also increase.

In general, primary blast injuries are characterized by the absence of external injuries; thus internal injuries are frequently unrecognized and their severity underestimated. According to the latest experimental results, the extent and types of primary blast-induced injuries depend not only on the peak of the overpressure, but also other parameters such as number of overpressure peaks, time-lag between overpressure peaks, characteristics of the shear fronts between overpressure peaks, frequency resonance, and electromagnetic pulse, among others. There is general agreement that implosion, inertia, and pressure differentials are the main mechanisms involved in the pathogenesis of primary blast injuries.

Thus, the majority of prior research focused on the mechanisms of blast injuries within gas-containing organs/organ systems such as the lungs, while primary blast-induced traumatic brain injury has remained underestimated. Blast lung refers to severe pulmonary contusion, bleeding or swelling with damage to alveoli and blood vessels, or a combination of these. Blast lung is the most common cause of death among people who initially survive an explosion. Applicants have surprisingly discovered that the shock waves and pressure waves that are known to harm organs and organ systems in a primary blast injury can be scaled down and controlled to provide systems and methods for controlled damage of cells and tissues (e.g., organs) that leads to improvement in the cells and tissues, improvements including tissue rejuvenation.

Laser Induced Optical Breakdown

Very short and high peak power a very short pulse width range from about 150 picoseconds to about 900 picoseconds, from about 200 picoseconds to about 500 picoseconds, or from about 260 to about 300 picoseconds comprised of deeply penetrating wavelengths (e.g., wavelengths such as that obtained with a 755 nm alexandrite laser and/or a 1064 nm NdYAG laser) may be focused at a depth in target tissues with the purpose of causing a laser induced optical breakdown (LIOB) injury. Additional details relating to LIOB formation by various lens arrays and their use in treatment methods are described herein.

FIG. 2 depicts a tissue injury 100 caused by the picosecond laser. At least a portion of the cavitation bubble 101 is ablated (e.g., vaporized) and in this pressure bubble 101 the photo thermal effect (e.g., temperature rise) of the picosecond laser on the tissue is largely confined. Biologic tissues and cells proximal to the surface of the cavitation bubble (ablation volume) therefore are exposed to the most intense shock wave region. Regions of tissues and cells farther from the cavitation bubble injury therefore are subject to ever decreasing pressure waves (e.g., ever decreasing magnitude pressure waves). This results in layers of cell damage not unlike layers of an onion, wherein layers of cells and tissue 102 more proximal to the cavitation bubble 101 experience the most intense pressure in shock waves and layers of cells and tissue more external to the bubble are subject to less intense pressure in pressure waves (e.g., cell layer 104 is exposed to less intense pressure than cell layer 103).

Referring still to FIG. 2, the injury 100 is comprised of a central cavitation bubble 101 at least a portion of which has an ablation volume surrounded by tissue regions of relatively high cellular damage 102 having the most damage outside the cavitation bubble 101 with tissue layer 102 having the most cell damage, for example, total damage and immediate cell death, which are in turn surrounded by tissue layers 103, 104 and 105 having progressively lower cellular damage such that longer term cell death occurs with each progressively outer layer. For example, tissue layer 103 having severe cell damage (e.g., from about 1 to about 2 days until cell death), tissue layer 104 having moderate cell damage (e.g., from about 2 to about 7 days until cell death), and tissue layer 105 having minor cell damage (e.g., from about 7 to about 21 days until cell death).

For example, layer 104 has a longer term cell damage (e.g., where cell death takes from about 2 to about 7 days) than layer 103 (e.g., where cell death takes from about 1 to about 2 days). The exemplary cell death dates are illustrative. Without being bound to any single theory, Applicants believe that it is important in that ongoing deaths of damaged cells which extend at least for several days and possibly for several weeks after the injury, are believed to enhance healing by continuing to deposit dead cell matter including proteins into nearby tissues. This ongoing long term cell death results in a longer duration of new cell genesis stimulated by the ongoing presence of cellular debris.

As the period of cell deaths extends, the period of presence of precursors for new cells is extended leading to a longer duration of stimulated new cell formation near the injury site, thereby improving healing and outcomes. It is believed that a sustained inflammatory period with ongoing release of cellular debris including cell proteins yields a longer period of new cell stimulation, a longer period of repair, and better healing compared, for example, to known photo thermal treatments (e.g., thermal laser treatments such as fractional photothermolysis).

The non-thermal effect (e.g., pressure wave and/or shock wave effect) of the cavitation bubbles are distinct from the pure photothermolysis effect resulting from laser irradiation. Photothermolysis does govern the underlying absorption of the applied laser pulse that forms the cavitation bubbles. Nevertheless, the non-thermal effects (e.g., shock waves and/or pressure wave and/or mechanical effects) are believed to create onion-like layers of lesions having varying amounts of cell damage within the target tissues.

Exemplary Lens Array, Shockwave induction, Sonoporation and Focal Technique Embodiments

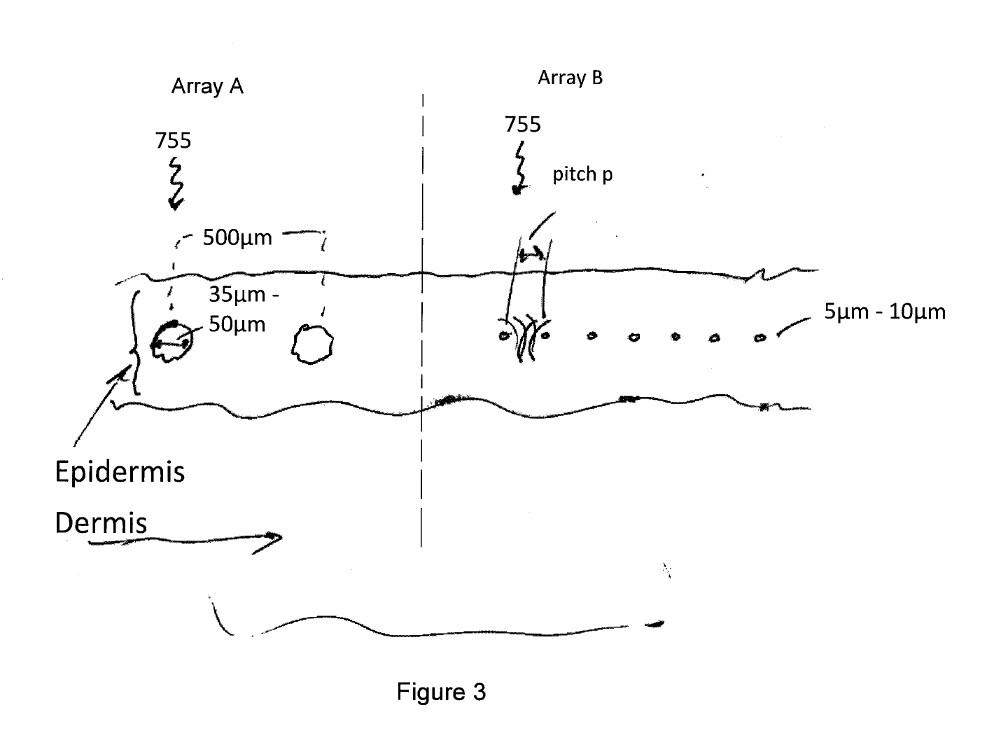

FIG. 3 shows an epidermis and dermis of subject that is being illuminated using two different lens arrays, array A and array B. FIG. 3 shows that lens array A and lens array B treat epidermis tissue (e.g., in the intra epidermal region) for example, tissue at a depth from the skin surface of from about 10 microns to about 90 microns, or from about 20 microns to about 80 microns, or from about 25 microns to about 75 microns. Alternatively or in addition lens array A and/or lens array B can be employed to treat the epidermal/dermal junctions and/or the dermis.

Each lens array A, B can be used to direct light pulses of various wavelengths suitable such as the exemplary 755 nm wavelength light shown directed towards the epidermis. Array A relates to a lens array that directs light pulses such as picosecond pulses to treatment regions that are separated by about 500 microns. The lens array provides a distance of about 500 μm between the centers of adjacent lenses for array A. The diameter of the treatment regions generated using array A ranges from about 35 microns to about 50 microns as shown in FIG. 3.

In array B of FIG. 3, shown on the right side of the figure, the pitch or separation distances between the pulses incident on the skin is sized to be less than about 500 μm. In one embodiment, the pitch is less than about 500 μm. In one embodiment, the pitch is less than about 300 μm. In one embodiment, the pitch is less than about 200 μm. In one embodiment, the pitch is less than about 100 μm. In one embodiment, the pitch is less than about 80μm. In one embodiment, the pitch is less than about 60 μm. The pitch is selected such that shockwaves induced by sonoporation resulting from incident pulses overlap. Thus, a lens array, such as array B, can be sized such that the incident pulses directed to the skin or other tissue result in overlapping sonoporation induced shockwaves. In one embodiment, the diameter of the treatment region on the skin or focal spots ranges from about 5 microns to about 10 microns as shown for array B.

The pitch p is shown in FIG. 3 in the region of overlapping shockwaves between a first treatment region and a second treatment region. This pitch p or separation distance can be selected such that the overlapping shockwaves that result that have a pressure less than 5 psi. This pitch p or separation distance can be selected such that the overlapping shockwaves that result have a pressure that ranges from about 1.5 psi to about 3 psi. This pitch p or separation distance can be selected such that the overlapping shockwaves that result have a pressure that ranges from about 8 Kpascal to about 18 Kpascal. These shockwaves are generated at a lower energy level. In turn, this results in a smaller focal spot, such as in the about 5 to about 10 micron range. This smaller focal spot, in turn, results in a smaller lesion on or in the skin. Similarly, a smaller lesion results in or corresponds to a small amount of tissue being ablated or necrotized which in turn results in shock wave induced sonoporation of cell membranes.

Cellular Membrane Porosity Related Embodiments

As noted herein, a lens array with a suitable pitch between focal spots and associated treatment regions can be used to produce tailored shockwaves. These shockwaves can be used to change membrane properties. In accordance with one embodiment, one exemplary use of the shockwave generating techniques described herein improves the cellulite membrane porosity of treated areas and allows cell membranes to uptake and engulf large molecules. This method may trigger gene expression or possibly “turn on” healing related genes in response to increased membrane porosity. In addition, in one embodiment, the light-based shockwave generation having the pressure characteristics described herein can be used to temporarily make membranes porous or more porous to allow bi-directional transport of intra and extra cellular material which otherwise would not occur. These membrane changes resulting from light-based shockwave generation can be used to facilitate cells to uptake large molecules such as cancer medications and other medicaments. In one embodiment, the sonoporation induced by shock waves allows free flow of material through cell walls temporarily which can be controlled and activated upon the firing of light pulses using a suitable array such as array B.

Channel Creation in Tissue Using Sequential or Stacked Laser Induced Optical Breakdown (LIOB) Pulses

In some applications it may be desirable to sequentially apply a series of stacked laser pulses. Each laser pulse is designed to individually exceed the LIOB threshold and cause plasma breakdown of the target area. For example, a laser pulse creates a LIOB ball whereby the region in focus is ablated and is surrounded by pressure treated regions. In one embodiment, a laser pulse initiated LIOB injury results in rapidly expanding bubbles. In some pressure regimes these rapidly expanding bubbles are cavitation bubbles. At least a portion of the tissue within rapidly expanding bubble (e.g., the cavitation bubble) is near-instantaneously vaporized providing an ablation volume. Adjacent the vaporized volume are a roughly spherical injury where the most intense pressure waves called shock waves are concentrated.

Shock waves are the first portion of a high pressure expansion that extend away from the surface of the cavitation bubble through proximal tissues and cells. The shock waves that initially emanate from the cavitation bubble attenuate as they propagate through proximal tissues and cells experiencing a reduction in pressure and velocity and are then referred to as pressure waves. The shock waves are pressure waves that travel faster than the speed of sound and are believed to exhibit non-linear behavior. Shock waves attenuate into pressure waves when they travel at the speed of sound or less than the speed of sound. The behavior creates regions of shock waves (and resulting relatively intense mechanical stress on tissue and/or intense cell damage) nearer the cavitation bubble and regions of relatively reduced intensity pressure waves (and relatively reduced mechanical stress on tissue and/or reduced cell damage) as the distance from the cavitation bubble increases.

As noted herein, as part of a light pulse-based method that sequentially applies a series of stacked laser pulses, each pulse can exceed the LIOB threshold and cause plasma breakdown of the target area of the epidermis and/or the dermis. The next stacked laser pulse will create a subsequent LIOB ball that will serve to further excavate and/or ablate material below the first cavity made by the first LIOB. As additional stacked pulses are generated, each subsequent LIOB is formed at or near the bottom of the previous cavity. Eventually the series of stacked laser pulses results in a channel forming through the tissue (e.g., in the z direction through the tissue area).

In one embodiment, light generated bubbles, in tissue, such as water containing tissue, can be used to provide additional pulse focusing. For example, after an LIOB pulse is initiated, a bubble expands and exists for a time period greater than about 0 to about 100 nanoseconds (or longer) in one embodiment. The application of a second laser pulse to the bubble generated by the first pulse is possible if transmitted within the time period the bubble exists, such as within 100 nanoseconds. The light generated bubble acts as a semi-spherical lens which acts to focus the second pulse deeper into the tissue using the temporarily formed lens. In one embodiment, an initial edge of a laser pulse, such as the leading (or trailing) edge of a pulse, generates the bubble in a target tissue or material, such as a water containing material, and the subsequent edge, such as a trailing (or leading) edge of laser pulse is focused by the bubble generated by the initial edge.

In one embodiment, the control system directs a second pulse through an LIOB bubble to act as a secondary focusing element when the LIOB the bubble is in a post-ionized state. LIOB bubbles, after the end of ionization, no longer absorb laser pulse energy. As a result, in a post-ionized state, in one embodiment, such bubbles may be used as secondary lenses for generating a subsequent LIOB at a location deeper in the tissue such as below at least a portion of the first LIOB region.

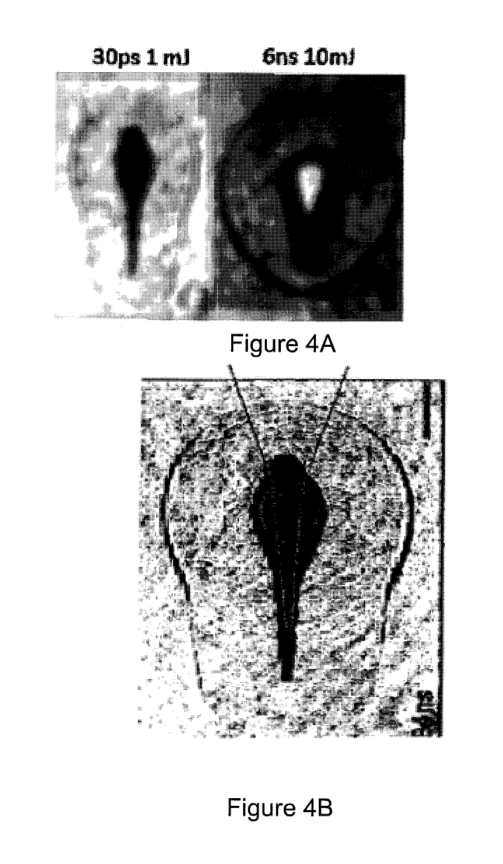

FIG. 4A shows an LIOB generated in water in response to a laser pulse with shockwaves. A 30 ps and 1 mJ pulse was used to generate the bubble shown on the left in water. A 60 ns and 10 mJ pulse was used to generate the bubble shown on the right in water. FIG. 4B shows another LIOB generated bubble in water in response to a laser pulse. In one embodiment, the trailing edge of a laser pulse is directed around the initial LIOB expansion region. The bubble shown can be fired through with a second pulse such as a picosecond pulse to focus deeper into a tissue or other material. The use of bubble-based focusing can result in smaller diameter channels and/or deeper penetration depths. In one embodiment, the bubbles are elongate or have a spiked shape.

While analogous to CO2 stacked ablative pulses common in aesthetic rejuvenation applications, LIOB excavated channels are mediated by a combination of ablated regions surrounded by pressure wave treated zones (e.g., shock waves that dissipate into pressure waves). In tissue treatment applications, in the ablated region of each LIOB ball is important because the size of the ablated region can be controlled to reduce the size of the diameter of the channels formed. In CO2 channel drilling applications tissue is vaporized by high linear absorption. Conversely, in a LIOB stacked pulse drilling application, tissue is vaporized by non-linear ionization. The advantage of stacked LIOB pulses being greater confinement of heat (LIOB and picosecond confinement of heat) such that area adjacent the channels are substantially free from temperature rise. Accordingly, channels created by a series of adjacent LIOB balls may have relatively smaller diameters and/or be capable of traveling to greater depths as compared to purely linear thermally mediated channels such as those created by CO2 or erbium 2940, for example. In one embodiment, channels created with stacked LIOB pulses are employed to improve the mobility limitations associated with certain mobility restricted scars (e.g., burn scars). In another embodiment, channels created with stacked LIOB pulses are employed to in orthopedic applications (e.g., to treat cartilage) by creating microfractures in the orthopedic tissue (e.g., in bones and/or in cartilage). In still another embodiment, channels created with stacked LIOB pulses are employed in cardiac applications (e.g., to treat heart tissue).

Sequential One Two Pulse:

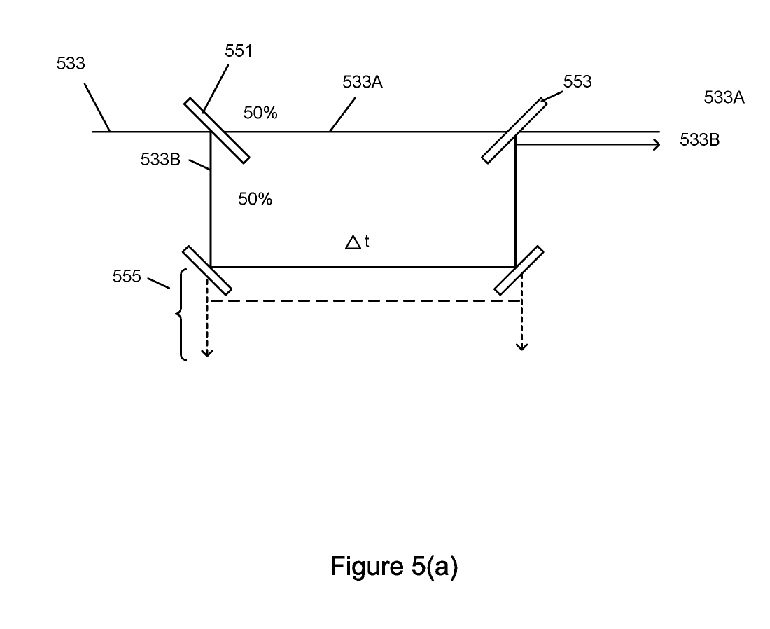

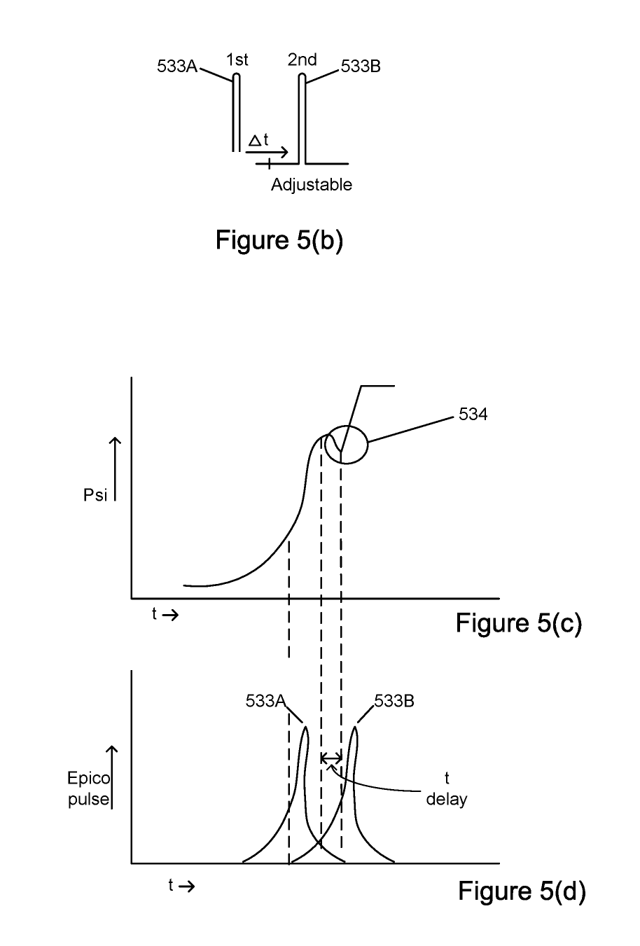

Referring now to FIGS. 5(a)-5(b), in a sequential one two pulse arrangement a picosecond drive pulse 533 is split by an adjustable beam splitter 551 into two equal parts 533A and 533B (e.g., 50% in the first part 533A and 50% in the second part 533B). One part (e.g., the second part 533B) can be delayed by an adjustable amount of time, therefore the first pulse 533A initiates LIOB (LIOB is not shown in this graphic) and the second pulse 533B arrives at the target while the target area is still ionized by the first pulse 533A, which previously initiated the LIOB. A second beam director 553 such as a beam splitter can also be use to further direct light as shown. Here, due at least in part to the delay, the second pulse 533B is immediately and fully absorbed by the target area and acts to drive a second pulse of expansion. The amount of delay between pulse 533A and 533B, shown as ΔT, can be adjusted by moving 555, which is a reflective or substantially reflective surface such a mirror.

Referring now to FIGS. 5(c)-5(d), in one embodiment there is a point where the peak pressure provided by the first pulse is just beginning to wane (but is still in a plasma/ionized state) then consequently the initial shockwaves generated by the first pulse will detach (such that it is no longer driven by the ablation bubble expansion) and will begin to propagate into the tissue just as the second pulse arrives, this is depicted in FIG. 5(c) as point 534 in a plot shown the time on the x-axis and the pressure in Psi on the y-axis. It is believed that this shockwave detachment will result in the second shockwave overtaking the initial shockwave wave front thereby providing an additive re-enforcement of the initial shockwave extending the range and the volume of pressure injured tissue.

Thus, the first LIOB is formed by a first pulse 533A and before it de-ionizes the LIOB is further driven to a second period of bubble expansion. This creates a second shockwave pulse 533B that, if sufficiently driven, can overtake and add to the first shockwave. The delayed second shockwave of the sequential one two pulse arrangement extends the volume of tissue treated with efficacious shockwaves. Applicants believe that the second pulse of energy benefit is larger than the first pulse, because the second shockwave pulse can catch up to and add to the first pulse wave front.

It is possible to adjust the energy provided in the first vs the second pulse (e.g., 533A vs 533B via the beam splitter) to tune the secondary wave front optimization. For example, first pulse may have less energy than the second pulse to support optimum wave front overtaking and additive pressure effects. In one embodiment, the one-two sequential firing can provide overlapping shots of the same target area. In another embodiment, the one-two sequential firing can have two adjacent target areas, for example.

A sequential one two pulse technique can provide an enhanced lesion. For example, a sequential one two pulse technique can optimize the shockwave effect by delivering a 2nd laser pulse to the target (LIOB expanded bubble) before ionization and non-linear absorption has discontinued. The technique initiates a second expanding shockwave to increase lesion size. FIGS. 5(a)-5(d) illustrate this approach. This is especially useful when delivering LIOB injuries as deeply as possible. This method allows for large ablation volumes, while keeping fluence through intervening tissue as low as possible (50/50 energy in shot 1 (e.g., 533A) and in shot 2 (e.g., 533B)).

In some embodiments, this sequential one-two pulse technique is paired with a micro-lens array to achieve a deeper reach, and is used as a method to increase ablated volume without increasing single pulse energy. Generally, ablated volume is proportionate to laser energy/pulse and this sequential one two pulse technique can achieve greater relative ablation volumes without increasing the applied energy than in the absence of using the sequential one two pulse technique. Suitable applications of the one two pulse technique alone and a lens array such as a quasi-parabolic 4 cell micro-lens array used alone or in combination can include treatment areas where a deep but also large lesion is desired.

Picosecond LIOB with a Fractional Beam Array

Use of a picosecond laser with an output beam modified via a fractional array (e.g., a micro-lens array that creates high intensity focal zones surrounded by non-treated or less treated area of tissue) or a non-uniform beam characterized by a cross-section corresponding to an array of relatively small, relatively high-fluence, spaced-apart regions superimposed on a relatively large, and relatively lower-fluence background provides thermal energy and mechanical energy that cause thermal injury and shockwave and/or pressure wave injury. Where the picosecond laser with the non-uniform array treats tissue there is a component of high fluence causing thermal damage. The regime of injury caused by heat is well known.

In contrast, the regime of injury effected by the combination of thermal energy and mechanical energy provided by the shockwaves and pressure waves resulting from treating tissue with a picosecond laser such as a PicoPulse™ laser with CAPS™ technology is new and is not yet well defined, however, applicant believes it is desirable to understand the effect on the tissue of these combined thermal and mechanical effects. The energies may happen at a different rate and at a varied combination. The balance of the thermal and/or mechanical energies may be controlled to achieve a desired tissue interaction/tissue effect.

Samples of tissue treated with a picopulse laser with a non-uniform array reviewed 3 months after treatment showed some elongation of elastic fibers. Under prior regimes for tissue treatment elastin elongation is not typically seen without a lot of thermal injury that leads to a great deal of downtime. Subject downtime limits treatment application to a relatively smaller group of subjects willing and able to devote time to recovery due to the obviousness of their treatment or to a smaller number of tissue sites that may be covered during the obvious recovery. Elastin elongation with minor to no downtime is surprising and desirable in that tissue treatment that leads to desired elastin elongation with less to no downtime opens up the treatment application to the larger population that can't afford downtime and to otherwise open tissue sites where obviousness signs of treatment dissuade treatment of the area.

It is understood in the prior art that in order to damage elastin a large thermal injury and/or a high temperature/fluence was required. Treatment with a picosecond laser was applied using a non-uniform beam array to achieve very high temperatures locally in a small area, this local thermal energy was combined with mechanical energy (e.g., shock wave and/or pressure wave energy).

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

While the invention has been described with reference to illustrative embodiments, it will be understood by those skilled in the art that various other changes, omissions and/or additions may be made and substantial equivalents may be substituted for elements thereof without departing from the spirit and scope of the invention. In addition, many modifications may be made to adapt a particular situation or material to the teachings of the invention without departing from the scope thereof. Therefore, it is intended that the invention not be limited to the particular embodiment disclosed for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

What is claimed is:

1.-20. (canceled)

21. A method for tissue treatment, comprising:
generating a first picosecond light pulse using a picosecond laser apparatus;
transmitting the first picosecond light pulse to a lens array;
directing a first plurality of picosecond light pulses from the lens array to a first plurality of treatment regions, wherein each such treatment region is excavated and/or ablated to form a first cavity therein;
generating a second picosecond light pulse using the picosecond laser apparatus;
transmitting the second picosecond light pulse to the lens array; and
directing a second plurality of picosecond light pulses from the lens array to a second plurality of treatment regions to form a second cavity therein,
wherein the second plurality of picosecond light pulses is stacked relative to the first plurality of picosecond light pulses such that each first cavity is further excavated and/or ablated by each second cavity formed below the first cavity.

22. The method of claim 21 further comprising repeating foregoing steps to form a third cavity below the second cavity such that a channel is formed from the first cavity, the second cavity, and the third cavity.

23. The method of claim 22, wherein channel is formed through the tissue in the z-direction.

24. The method of claim 21 wherein each cavity is formed by a LIOB injury to the tissue.

25. The method of claim 21, wherein the lens array comprises two or more foci for concentrating the pulses at a fluence sufficient to create electron ionization of the two or more stacked treatment regions.

26. The method of claim 21, wherein the pulse width is within the range of from about 260 picoseconds to about 900 picoseconds.

27. The method of claim 26 further comprising tuning the pulse width to change ratio of pressure wave to a thermal effect for tissue adjacent a treatment region.

28. The method of claim 22 further comprising repeating foregoing steps to increase depth of channel in the z-direction.
